(19) United States
(12) Patent Application Publication
(10) Pub. No.: US 2025/0161530 A1
(43) Pub. Date: May 22, 2025

(54) NOVEL ELECTROSPUN SYNTHETIC MEMBRANES FOR SOFT TISSUE REPAIR APPLICATIONS

(71) Applicant: Matregenix, Inc., Mission Viejo, CA (US)

(72) Inventor: Sherif Soliman, Irvine, CA (US)

(73) Assignee: Matregenix, Inc., Mission Viejo, CA (US)

(21) Appl. No.: 19/032,033

(22) Filed: Jan. 18, 2025

Related U.S. Application Data
(63) Continuation-in-part of application No. PCT/US2023/028752, filed on Jul. 26, 2023.
(63) Continuation-in-part of application No. 16/910,051, filed on Jun. 23, 2020.
Application No. 16/910,051 is a continuation-in-part of application No. PCT/US2018/067427, filed on Dec. 23, 2018.
(60) Provisional application No. 63/369,418, filed on Jul. 26, 2022.
(60) Provisional application No. 62/610,155, filed on Dec. 23, 2017.

(51) Int. Cl. A61L 27/18; A61L 27/54; A61L 27/56; B32B 5/02; B32B 27/36

(52) U.S. Cl. CPC A61L 27/18; A61L 27/54; A61L 27/56; B32B 5/022; B32B 27/36; A61L 2420/02; A61L 2430/34; B32B 2262/0276; B32B 2535/00

(57) ABSTRACT

The present disclosure describes membranes suitable for use in soft tissue repair applications that are composed of fibrous and highly porous biodegradable materials fabricated using electrospinning and that may be surface-modified with plasma treatment or other suitable methods of surface modification. The disclosed membranes have a high surface area to volume ratio. In some implementations, use of the disclosed membranes provides a barrier that prevents the migration of soft tissue cells but is permeable to small molecules such as nutritional substances and medications. In other implementations, use of the disclosed membranes provides a scaffold to facilitate soft tissue repair by providing a suitable environment for cellular infiltration and interaction to promote tissue regeneration. Methods of fabricating the disclosed resorbable membranes for soft tissue repair applications using electrospinning are also disclosed. The disclosed membranes may have precisely tuned physical, chemical, and mechanical properties optimized for various soft tissue repair applications.

### CROSS-REFERENCE TO RELATED APPLICATIONS

This application is a continuation-in-part of PCT Patent Application Serial No. PCT/US2023/028752, filed on Jul. 26, 2023, which claims the benefit of U.S. Provisional Patent Application Ser. No. 63/369,418, filed on Jul. 26, 2022; and this application is also a continuation-in-part of U.S. patent application Ser. No. 16/910,051, filed on Jun. 23, 2020, which is a continuation-in-part of PCT Patent Application Serial No. PCT/US2018/067427, filed on Dec. 23, 2018, which claims the benefit of U.S. Provisional Patent Application Ser. No. 62/610,155, filed on Dec. 23, 2017, the disclosures of which are hereby incorporated herein in their entireties by reference.

### BACKGROUND

### Field of the Invention

The present disclosure relates to synthetic membranes for soft tissue repair applications.

### Description of the Related Art

Soft tissue repair is an area of interest for a variety of applications, including applications in dentistry, skin repair, orthopedics, abdominal surgery, dural repair, chest wall reconstruction, hernia repair, tissue reinforcement, and other areas.

For example, regeneration of lost periodontal tissues requires the use of barrier dental membrane devices to prevent soft-tissue invasion into the defect and guide the bone regeneration process. See Dimitriou, R., et al. “The Role of Barrier Membranes for Guided Bone Regeneration and Restoration of Large Bone Defects: Current Experimental and Clinical Evidence,” BMC Med. 2012, 10 (81), doi: 10.1186/1741-7015-10-81. Biocompatibility, space-making ability, the ability to achieve tissue integration, and clinical manageability are criteria that must be considered in the design of materials used for regenerative procedures. See Scantlebury, T. “1982-1992: A Decade of Technology Development for Guided Tissue Regeneration,” J. Periodontol. 1993, 64, 11-29.

A number of barrier membranes have been used to achieve the desired reconstruction, but each type of barrier membrane that has been used falls short of satisfying all of the aforementioned criteria. The current available membranes may be classified as non-resorbable or resorbable. Non-resorbable membranes have the disadvantage of requiring a second surgery to remove the membrane, which often carries a risk of infection and patient discomfort. The application of non-resorbable membranes requires a high level of surgical skill to trim and shape the membrane for use, and the use of non-resorbable membranes has exhibited an unacceptable degree of failure. See Bottino, M. C., et al. “Recent Advances in the Development of GTR/GBR Membranes for Periodontal Regeneration-A Materials Perspective,” Dent. Mater. 2012, 28 (7), 703-21. The majority of currently available resorbable dental membranes are composed of collagen, on account of its excellent biocompatibility. While the use of a collagen membrane eliminates the need for a second surgery, collagen membranes typically absorb at a higher rate than ideally needed to maintain the needed physical space for regeneration and to achieve surgical goals. See Rodella, L. F., et al. “Biomaterials in Maxillofacial Surgery: Membranes and Grafts,” Int. J. Biomed. Sci. 2011, 7 (2), 81-88. In addition, animal derivative materials such as collagen present problems of rejection, variability, and insufficient mechanical strength. See Dori, F., et al. “Effect of Platelet-Rich Plasma on the Healing of Intra-Bony Defects Treated with a Natural Bone Mineral and a Collagen Membrane,” J. Clin. Periodontol. 2007, 34 (3), 254-61. More recently, synthetic membranes have been used to address the limitations associated with collagen membranes. Currently available synthetic membranes are based on lactides, glycolides, and lactones. The resorption mechanism for the available synthetic membrane materials is typically limited to enzymatic and hydrolytic processes, which results in local acidification of tissue, thereby causing inflammation and bone erosion. See Azevedo, H. S., et al. “Understanding the Enzymatic Degradation of Biodegradable Polymers and Strategies to Control Their Degradation Rate,” In Biodegradable Systems in Tissue Engineering and Regenerative Medicine, Reis, et al. Eds., 2004, 177-201. The currently available synthetic membranes degrade via a bulk erosion mechanism, which makes it very difficult to predict the degradation rate of the membranes. Id. Moreover, studies show that the use of currently available synthetic membranes leads to epithelial downgrowth, gingival recession, device exposure, and pronounced soft tissue inflammation. See Laurell, L., et al. “Gingival Response to GTR Therapy in Monkeys Using Two Bioresorbable Devices (Abstract 824),” J. Dent. Res. 1993, 72, 206. As a result, these membranes are not widely used in dental clinics. It is thus clear that the “ideal” membrane for use in periodontal regenerative therapy has yet to be developed.

The generation of a membrane that overcomes all of the aforementioned structural, mechanical, and bio-functional limitations in dental applications and analogous challenges in other applications would have a significant positive impact on the field of soft tissue repair.

Recently developed methods of scaffolding for tissue engineering offer promise in this regard. Electrospinning is a process that uses an electric field to generate continuous fibers on a micrometer or nanometer scale. Electrospinning has been shown as a promising method for the fabrication of tissue engineering scaffolds, as the resultant structures mimic the topology of the native extracellular matrix. See, e.g., Li, W. J., et al. “Electrospun Nanofibrous Structure: A Novel Scaffold for Tissue Engineering,” J. Biomed. Mater. Res. 2002, 60 (4), 613-21; Pham, Q. P., et al. “Electrospinning of Polymeric Nanofibers for Tissue Engineering Applications: A Review,” Tissue Eng. 2006, 12 (5), 1197-211; Murugan, R., et al. “Nano-Featured Scaffolds for Tissue Engineering: A Review of Spinning Methodologies,” Tissue Eng. 2006, 12 (3), 435-47. Electrospinning enables direct control of the microstructure of a scaffold, including characteristics such as the fiber diameter, orientation, pore size, and porosity. Electrospinning has been extensively investigated as a technique for producing tunable scaffolds for various tissue engineering applications. It is believed that electrospun fibers are effective as tissue regenerative scaffolds because of their ability to mimic the fibrous extra-cellular matrix (ECM) of human tissues. However, the use of electrospun scaffolds also presents challenges for many tissue engineering applications. For example, it is difficult to promote cellular penetration into the depth of an electrospun structure, despite the porosity and flexibility afforded by the electrospinning process. The small average pore size provided by the non-woven fibrous dense structure often leads to a preference for cells to proliferate and migrate across the surface of the scaffold rather than inside it.

Synthetic biomaterials have gained substantial interest in various tissue engineering applications, on account of the physical and biological properties of such materials. See, e.g., Yoshimoto, H., et al. “A Biodegradable Nanofiber Scaffold by Electrospinning and Its Potential for Bone Tissue Engineering,” Biomaterials, 2003, 24, 2077; Albertsson, A.-C., et al. “Recent Developments in Ring Opening Polymerization of Lactones for Biomedical Applications,” Biomacromolecules, 2003, 4, 1466; Hutmacher, D. W., et al. “Mechanical Properties and Cell Cultural Response of Polycaprolactone Scaffolds Designed and Fabricated via Fused Deposition Modeling,” J. Biomed. Mater. Res. 2001, 55, 203; Li, W.-J., et al. “Biological Response of Chondrocytes Cultured in Three-Dimensional Nanofibrous Poly (&-caprolactone) Scaffolds,” J. Biomed. Mater. Res. A, 2003, 67A, 1105; Lin, W.-J., et al. “A Novel Fabrication of Poly (&-caprolactone) Microspheres from Blends of Poly (&-caprolactone) and Poly (ethylene glycol) s,” Polymer, 1999, 40, 1731; Zong, X., et al. “Structure and Morphology Changes During In Vitro Degradation of Electrospun Poly (glycolide-co-lactide) Nanofiber Membrane,” Biomacromolecules, 2003, 4, 416; Kim, C. H., et al. “An Improved Hydrophilicity via Electrospinning for Enhanced Cell Attachment and Proliferation,” J. Biomed. Mater. Res. B Appl. Biomater. 2006, 78B, 283. Polymeric synthetic biomaterials have been shown to degrade mainly by simple hydrolysis of the polymer backbone bonds into acidic monomers, which are subsequently removed from the body by normal metabolic pathways. However, most polymeric synthetic biomaterials have a hydrophobic surface, thus limiting the applications in which they may be used. In addition, most polymeric synthetic biomaterials are also known for their slow degradation rate due to the presence of a hydrophobic surface that thus retards hydrolysis.

Electrospinning of biomaterials such as polycaprolactone, polylactic-co-glycolic acid, and chitosan has been used to generate guided tissue regeneration (GTR) and guided bone regeneration (GBR) barrier membranes. See, e.g., Xue, J., et al. “Electrospun Microfiber Membranes Embedded with Drug-Loaded Clay Nanotubes for Sustained Antimicrobial Protection,” ACS Nano, 2015, 9 (2), 1600-12; Carter, P., et al. “Facile Fabrication of Aloe Vera Containing PCL Nanofibers for Barrier Membrane Application,” J. Biomater. Sci. Polym. Ed. 2016, 27 (7), 692-708, doi: 10.1080/09205063.2016.1152857; Yang, F., et al. “Development of an Electrospun Nano-Apatite/PCL Composite Membrane for GTR/GBR Application,” Acta Biomater. 2009, 5, 3295-3304, doi: 10.1016/j.actbio.2009.05.023; Jia, J., et al. “Preparation and Characterization of Soluble Eggshell Membrane Protein/PLGA Electrospun Nanofibers for Guided Tissue Regeneration Membrane,” J. Nanomater. 2012, doi: 10.1155/2012/282736; Qasim, S. B., et al. “Potential of Electrospun Chitosan Fibers as a Surface Layer in Functionally Graded GTR Membrane for Periodontal Regeneration,” Dent. Mater. 2017, 33 (1), 71-83. The use of copolymers, composites, and blends of the biomaterials such as polycaprolactone, polylactic-co-glycolic acid, and chitosan results in GTR and GBR barrier membranes with properties that are better suited for the desired applications. See, e.g., Liao, S., et al. “A Three-Layered Nano-Carbonated Hydroxyapatite/Collagen/PLGA Composite Membrane for Guided Tissue Regeneration,” Biomaterials, 2005, 26 (36), 7564-71; Bottino, M. C., et al. “A Novel Spatially Designed and Functionally Graded Electrospun Membrane for Periodontal Regeneration,” Acta Biomater. 2011, 7 (1), 216-24. These electrospun GTR and GBR barrier membranes are nonetheless limited by the properties of the biomaterials used.

There remains a significant need for membranes for use in soft tissue repair applications that combine the advantages of currently available synthetic materials with respect to stability and mechanical properties and the advantages of natural materials with respect to biocompatibility, thereby overcoming the structural, mechanical, and bio-functional limitations of currently available membranes.

### SUMMARY

The present disclosure describes membranes suitable for use in soft tissue repair applications that are composed of fibrous and highly porous biodegradable materials fabricated using electrospinning and that may be surface-modified with plasma treatment or other suitable methods of surface-modification. The disclosed membranes have a high surface area to volume ratio. In some embodiments, use of the disclosed membranes provides a barrier that prevents the migration of soft tissue cells but is permeable to small molecules such as nutritional substances and medications. In other embodiments, use of the disclosed membranes provides a scaffold to facilitate soft tissue repair by providing a suitable environment for cellular infiltration and interaction to promote tissue regeneration. Methods of fabricating the disclosed resorbable membranes for use in soft tissue repair applications using electrospinning are also disclosed. Electrospinning allows precise control over the pore size and microstructure characteristics of the membranes generated. The disclosed membranes may have precisely tuned physical, chemical, and mechanical properties optimized for various soft tissue repair applications.

### BRIEF DESCRIPTION OF THE DRAWINGS

FIG. 1A shows representative SEM micrographs of a bilayer membrane.

FIG. 1B shows fiber diameter distribution for the bilayer membrane of FIG. 1A.

FIG. 1C shows porosity measurements for the bilayer membrane of FIG. 1A.

FIG. 1D shows pore size results for the bilayer membrane of FIG. 1A.

FIG. 2 shows the results of wetting analysis testing via a DCA wicking experiment, showing the difference in total normalized weight gain during immersion between the pre-treated and post-treated samples.

FIG. 3 shows representative tensile testing results of pre- and post-treated samples, including the stress-strain curve in FIG. 3A, the load at break in FIG. 3B, the tensile strain at break in FIG. 3C, and the Young's modulus in FIG. 3D.

### DETAILED DESCRIPTION

The present disclosure describes membranes suitable for use in soft tissue repair applications that are composed of fibrous and highly porous biodegradable materials fabricated using electrospinning and that may be surface-modified with plasma treatment or other suitable methods of surface-modification. In some embodiments, use of the disclosed membranes provides a barrier that prevents the migration of soft tissue cells but is permeable to small molecules such as nutritional substances and medications. Permeability to small molecules results from the high surface area to volume ratio of the disclosed electrospun biodegradable materials. In other embodiments, use of the disclosed membranes provides a scaffold to facilitate soft tissue repair by providing a suitable environment for cellular infiltration and interaction to promote tissue regeneration.

As used herein, the term “membrane” refers to a thin, pliable sheetlike structure that may act as a boundary, lining, barrier, or partition, may alternatively act as a matrix or scaffold, or may combine these features. In applications where a membrane acts as a matrix or scaffold, it may be referred to as a membrane, a matrix, a scaffold, or a combination of these terms. A membrane has two surfaces, which may be referred to as the top surface and the bottom surface, the inner surface and the outer surface, or according to other suitable designations of the surfaces. A membrane also has a thickness, corresponding to the orthogonal distance between the surfaces.

As used herein, the terms “pore size” and “pore diameter” are used interchangeably.

The term “about” when used as a modifier is intended to convey that the numbers and ranges disclosed herein may be flexible as understood by ordinarily skilled artisans and that practice of the disclosed invention by ordinarily skilled artisans using properties that are outside of a literal range will achieve the desired result.

In some embodiments, a method of fabricating a resorbable membrane for soft tissue repair applications using electrospinning is disclosed. Electrospinning allows precise control over the pore size and microstructure characteristics of the membranes generated using the disclosed methods. The soft tissue repair applications may include but are not limited to dental applications, such as maxillofacial tissue reconstruction and guided tissue regeneration (GTR) and guided bone regeneration applications, including socket preservation, ridge augmentation, sinus lift, treating periodontal defects, and implant dehiscence; skin repair applications such as repair of skin burns and repair of both acute and chronic partial and full thickness wounds; orthopedic applications such as rotator cuff repair and tendon repair; abdominal surgery applications such as post-surgical adhesion barriers; dural repair applications; chest wall reconstruction applications; hernia repair applications such as diaphragmatic hernia repair and ventral hernia repair; and tissue reinforcement applications.

In some embodiments, the disclosed methods may be used to generate membranes with precisely tuned physical properties. In some embodiments, the disclosed methods may be used to generate membranes with precisely tuned chemical properties. In some embodiments, the disclosed methods may be used to generate membranes with precisely tuned mechanical properties.

In some embodiments, the disclosed methods may be used to generate membranes with precisely tuned physical and chemical properties. In some embodiments, the disclosed methods may be used to generate membranes with precisely tuned physical and mechanical properties. In some embodiments, the disclosed methods may be used to generate membranes with precisely tuned chemical and mechanical properties.

In some embodiments, the disclosed methods may be used to generate membranes with precisely tuned physical, chemical, and mechanical properties.

The disclosed membranes may be fabricated using various synthetic or natural materials including amino acid-based poly (ester urea) (PEU), polydioxanone (PDO), polylactic-co-glycolic acid (PLGA), polylactic acid (PLA), polycaprolactone (PCL), 4-hydroxybutyrate (4HB), poly 4-hydroxybutyric acid (P4HB), chitosan, silk, or combinations thereof. In some embodiments, the amino acid-based poly (ester urea) may include one or more amino acids selected from the group consisting of L-leucine, L-isoleucine, L-valine, and L-phenylalanine. One example of a suitable PEU material is L-valine-co-L-phenylalanine poly (ester urea). This PEU material does not produce any acidic byproducts that would trigger inflammation when a membrane comprising said PEU material is used as a barrier membrane or scaffold.

Electrospinning may be performed using any known electrospinning setup suitable for electrospinning polymer fibers. In some embodiments, an electrospinning apparatus comprising a syringe pump, a syringe, a power supply, and a mandrel or drum for fiber collection is used to electrospin a polymer into electrospun polymer fibers to generate an electrospun construct. Electrospinning may be carried out in a single step or multiple steps. The electrospun construct may preferably be a membrane. In some embodiments, the polymer is dissolved in a solvent to generate a polymer solution, the polymer solution is added to the syringe, and the syringe is then loaded into the syringe pump prior to electrospinning of the polymer fibers.

In some embodiments, the solvent may be hexafluoroisopropanol (HFIP), dichloromethane, methanol, tetrahydrofuran (THF), acetone, chloroform, water, phosphate-buffered saline (PBS), or a combination thereof.

Although the non-inflammatory characteristics of L-valine-co-L-phenylalanine poly (ester urea) render it highly suitable for use in tissue repair applications, membranes composed solely of electrospun nanofibers of L-valine-co-L-phenylalanine poly (ester urea) are prone to shrinkage and hardening upon exposure to human physiological conditions. This may be overcome by incorporating a stabilizing polymer, such as polydioxanone (PDO) or another suitable polymer such as polylactic-co-glycolic acid (PLGA), polylactic acid (PLA), polycaprolactone (PCL), 4-hydroxybutyrate (4HB), poly 4-hydroxybutyric acid (P4HB), chitosan, or silk, or combinations thereof, into the membranes. Incorporation of the stabilizing polymer may be achieved by electrospinning a solution of L-valine-co-L-phenylalanine poly (ester urea) and the stabilizing polymer in a suitable solvent, such as HFIP.

It has been shown that electrospun nanofibrous membranes composed of a blend of L-valine-co-L-phenylalanine poly (ester urea) and PDO do not shrink and also maintain dimensional stability in tissue culture media that mimic human physiological conditions.

In some embodiments, the ratio of L-valine-co-L-phenylalanine poly (ester urea) to the stabilizing polymer may be about 10:1 weight/weight. In some alternate embodiments, the ratio of L-valine-co-L-phenylalanine poly (ester urea) to the stabilizing polymer may be about 2:1 weight/weight. In some other alternate embodiments, the ratio of L-valine-co-L-phenylalanine poly (ester urea) to the stabilizing polymer may be about 4:3. Other weight/weight ratios of L-valine-co-L-phenylalanine poly (ester urea) to the stabilizing polymer may be used as optimized for the specific application in which the membrane is used.

In some embodiments, the electrospun membrane generated using the disclosed methods may be composed of a single layer or multiple integrated layers. In some embodiments, the membrane generated may be composed of multiple integrated layers with distinguishable microstructure characteristics.

In some embodiments, the nanofibers that compose the membranes are electrospun with two different electrospinning setups used at the same time. One setup may be a nozzle-based setup and the other setup may be a free surface needle-less slit setup. A single polymer solution may be used, or alternatively two separate polymer solutions may be used. This dual setup produces a hierarchical structure with two intermingled fiber diameters-smaller fibers from the nozzle setup and larger fibers from the needle-less setup-thereby increasing the total surface area and cell infiltration ability of the nanofibrous membrane.

In some embodiments, two distinct combinations are simultaneously subjected to electrospinning. The first combination is composed of L-valine-co-L-phenylalanine poly (ester urea) and PDO, while the second combination is composed of a sacrificial water-soluble polymer like poly (vinyl alcohol) (PVA), polyvinyl butyral (PVP), or B-silk. Following the electrospinning process, the resulting structure may be submerged in water, causing the water-soluble polymer to dissolve. This dissolution generates a membrane with a higher surface area, which ultimately facilitates superior cellular infiltration.

As described herein, the disclosed membranes may be used as barrier membranes to prevent soft tissue invasion as desired in applications such as dental barrier membranes and adhesion barriers for post-abdominal surgeries. Alternatively, the disclosed membranes may be used as scaffolds or matrices to facilitate soft tissue regeneration as desired in a variety of other soft tissue repair applications. Where the membrane acts as a scaffold or matrix, the membrane sufficiently resembles the native extracellular matrix to provide a suitable environment for cellular infiltration and interaction to encourage tissue regeneration.

Whether a membrane has barrier or scaffold functionality may be determined by average pore size. Barrier membranes typically have smaller fiber diameters, and thus smaller average pore sizes. The mean pore size for barrier membranes is smaller than the size of cells against which the barrier function is desired. By contrast, membranes acting as scaffolds typically have larger fiber diameters, and thus larger average pore sizes. Using a membrane as a scaffold requires cellular infiltration, and thus the mean fiber diameter must be large enough to allow cellular penetration and/or infiltration within the depth of the membrane.

In some embodiments, the membrane may be a scaffold or matrix for soft tissue repair that mimics the topology of the extracellular matrix. In some such embodiments, the membrane may be composed of a hierarchy of non-woven mesh structures.

In some implementations, the fiber diameter of the electrospun polymeric fibers may be between about 100 nm and about 15 μm.

In some implementations, the fiber diameter of the electrospun polymeric fibers may be between about 500 nm and about 2 μm. Contrary to prior suggestions by others that an ideal fiber diameter for tissue-engineered scaffolds ranges between 20 nm and 500 nm, it was shown that fiber diameters between 500 nm and 2 μm are necessary for proper cell infiltration into the depth of the electrospun matrices, thereby achieving host tissue integration in vivo.

In some implementations, the thickness of the membrane may be between about 100 μm and 2 mm. In some preferred implementations, the membrane may be composed of at least two layers with different average pore sizes. This may preferably enhance the functionality of the membrane as a barrier for soft tissue that promotes healing. The layer with the smaller average pore size may preferably function as a barrier during tissue healing, preventing soft tissue infiltration into a bone defect in certain applications and also stabilizing one or more blood clots formed during healing. The layer with the larger average pore size may preferably promote cell infiltration and, in certain applications, guided bone healing.

In some implementations, the membrane may be composed of two or more polymer layers, where the two or more polymer layers are each composed of a polymer or a combination of polymers. The two or more polymer layers include at least two distinct layers, including a layer having pores with a smaller average pore diameter and a layer having pores with a larger average pore diameter. The porosities of the two distinct layers are similar. The porosities of the two distinct layers may preferably differ by less than 5%.

In some such implementations, the layer having pores with a smaller average pore diameter has an average pore diameter of about 5-15 μm and is composed of polymer fibers having at least 95% of the polymer fibers with fiber diameters of about 0.1-2 μm, more preferably having at least 95% of the polymer fibers with fiber diameters of about 0.5-2 μm. In such implementations, the layer having pores with a larger average pore diameter has an average pore diameter of about 35-60 μm and is composed of polymer fibers having at least 95% of the polymer fibers with fiber diameters of about 3-7 μm. The porosities of both the layer having pores with a smaller average pore diameter and the layer having pores with a larger average pore diameter is about 80-95%, and the porosities of the two layers differs by less than 5%.

In some preferred implementations, the membrane may be composed of three layers, where one layer has a small average pore size and two layers have a large average pore size and the layer that has a small average pore size is situated between the two layers that have a large average pore size. In such implementations, the small average pore size is different from the large average pore size. This configuration may enhance integration of the layer that has a small average pore size within the membrane and may significantly reduce the risk of delamination. In some implementations, the small average pore size may be between about 1-20 μm and the large average pore size may be between about 20-400 μm. In some implementations, the average pore size of a layer may be determined by adjusting the viscosity of the polymer solution and adjusting the electrospinning process conditions to stabilize the spinning jet. Solutions with lower viscosity may be used to produce layers having a small average pore size, and solutions with higher viscosity may be used to produce layers having a large average pore size.

In some implementations, the mechanical integrity and binding forces between layers of the membrane may be enhanced by electrospraying short fibers prior to electrospinning the subsequent layer. In some other implementations, the mechanical integrity and binding forces between layers of the membrane may be enhanced by electrospinning wet fibers by decreasing the screen distance to generate a “tacky surface” prior to electrospinning the subsequent layer.

In some preferred implementations, a tubular braided structure or collapsible sleeve may be applied to the mandrel prior to electrospinning. The braid or sleeve may be metal or plastic. The braid or sleeve may facilitate the release of the electrospun construct from the mandrel. The use of a braid or sleeve may prevent damage to the morphology of the electrospun fibers during release from the mandrel.

In some preferred implementations, residual solvent may be removed from the electrospun construct by heating the electrospun construct to a temperature below the glass transition temperature of the polymer in a convection oven or a vacuum oven. In some implementations, residual solvent may be removed from the electrospun construct by immersing the electrospun construct in a solvent other than the residual solvent, whereby the residual solvent is removed from the electrospun construct via a liquid-liquid exchange mechanism. The solvent used to remove residual solvent may preferably be methanol. In some implementations, residual solvent may be removed from the electrospun construct by heating the electrospun construct to a temperature below the glass transition temperature of the polymer in a convection oven or a vacuum oven and also separately immersing the electrospun construct in a solvent other than the residual solvent to remove the residual solvent via a liquid-liquid exchange mechanism. The residual solvent may preferably be removed to the extent that after the solvent removal the electrospun construct contains an amount of solvent that is less than physiologically acceptable tolerance limits for the solvent.

In some preferred implementations, the surface chemistry of the electrospun membrane may be altered to enhance one or more properties including the wettability, conformability during use in soft tissue repair, and host tissue interactions of the membrane. The alteration of surface chemistry may be achieved by post-process treatment of the electrospun membrane or pre-process blending of an additional component into the polymer solution that is electrospun. The surface chemistry of the electrospun membrane may be altered using one or more methods selected from the group consisting of plasma treatment of the electrospun membrane with one or more gases and blending the polymer solution with a non-ionic surfactant prior to electrospinning.

In some preferred implementations, the gas used for plasma treatment may be introduced at a low pressure. In some implementations, the plasma treatment may comprise treatment with at least one mixture of more than one gas. In some implementations, the plasma treatment may comprise multiple separate and sequential treatment cycles comprising a first treatment cycle and a second treatment cycle, where the first treatment cycle comprises treatment with a first gas and the second treatment cycle comprises treatment with a second gas, where the first gas and second gas may be a single gas or a mixture of gases, and where the first gas differs in composition from the second gas. In some implementations, the gas may be one or more gases selected from the group consisting of oxygen, nitrogen, argon, and ethylene oxide.

In some implementations, the non-ionic surfactant may be selected from the group consisting of Pluronic-F108 and Pluronic-F127. In some implementations, the non-ionic surfactant may be Pluronic-F108. In some implementations, the non-ionic surfactant may be Pluronic-F127.

Incorporating a non-ionic surfactant like Pluronic F-108 or Pluronic F-127 into the polymer blend increases the surface hydrophilicity of membranes generated therefrom and provides better conformability and drapability when applied to defected tissue. Although Pluronic surfactants affect cell adhesion negatively, when added at a low ratio (10:1 wt/wt polymer to surfactant), the resulting membrane is hydrophilic without compromising cell adhesion.

In some preferred implementations, an additive or coating material may be added to the polymer solution or physically coated on the surface of electrospun membranes after electrospinning. The additive or coating material may be one or more additives or coating materials selected from the group consisting of platelet rich plasma (PRP), fibroblast growth factor (bFGF), hydroxyapatite, calcium phosphate, metronidazole (MNA), and N-methylpyrrolidone (NMP).

In some preferred implementations, the surface of the electrospun membrane may be coated with an adhesive so that the membrane may be applied to a surgical site without suturing. The adhesive may be a biodegradable synthetic adhesive or a natural polymer. The adhesive may be one or more adhesives selected from the group consisting of poly (dopamine), fibrin glue, elastin, dihydroxyphenylalanine (DOPA) derivatives, polyethylene glycol (PEG), hyaluronic acid, polyethylene glycol (PEG) and its derivatives, alginate, calcium, gelatin, chitosan, polysaccharides, and poly amido amine (PAMAM) dendrimer.

In some preferred implementations, an oxygen plasma treatment may be used to activate the surface of the electrospun membrane prior to coating with an adhesive. The activation of the surface of the electrospun membrane using oxygen plasma treatment will generate functional groups such as hydroxyls on the surface of the membrane to facilitate adhesion to the adhesive via a click chemistry mechanism.

In some implementations, the electrospun membrane may be sized into a size that is suitable for use in dental applications using laser cutting and printing. In some implementations, the membranes may be marked to identify a top side and a bottom side.

In some implementations, the electrospun membrane may be sterilized using electron beam or gamma sterilization procedures.

In some preferred implementations, the disclosed membranes possess excellent mechanical strength. This may facilitate suture retention in various soft tissue repair applications. In addition, a mechanically sound membrane with sufficient load-bearing ability will be able to maintain a suitable physical space for the intended soft tissue repair.

In some preferred implementations, membranes generated using the disclosed methods are malleable. This may facilitate manipulation of the membranes to assume the specific geometry required to maximize functionality of the tissue repair or reconstruction in a specific application.

In some preferred implementations, the disclosed membranes are fully resorbable when used in soft tissue repair applications in humans. Resorbability is achieved via degradation of the membrane, and this obviates the need for a second surgery to remove a membrane used in a soft tissue repair application.

In some implementations, the degradation rate of the membrane may be adjusted by adjusting the fiber diameter and thereby changing the total surface area to volume ratio, by adjusting the thickness of the membrane, or by adjusting both the fiber diameter and the thickness of the membrane.

In some implementations, the ratio of L-valine to L-phenylalanine in membranes composed of L-valine-co-L-phenylalanine poly (ester urea) or L-valine-co-L-phenylalanine poly (ester urea) blended with one or more stabilizing polymers may be adjusted, resulting in a membrane with adjustable mechanical strength and resorption rates ranging from one month to one year. In some implementations, the ratio of L-valine to L-phenylalanine in membranes composed of L-valine-co-L-phenylalanine poly (ester urea) or L-valine-co-L-phenylalanine poly (ester urea) blended with one or more stabilizing polymers is about 30:70. In some alternate implementations, the ratio of L-valine to L-phenylalanine in membranes composed of L-valine-co-L-phenylalanine poly (ester urea) or L-valine-co-L-phenylalanine poly (ester urea) blended with one or more stabilizing polymers is about 50:50. In some other alternate implementations, the ratio of L-valine to L-phenylalanine in membranes composed of L-valine-co-L-phenylalanine poly (ester urea) or L-valine-co-L-phenylalanine poly (ester urea) blended with one or more stabilizing polymers is about 70:30. Other L-valine to L-phenylalanine ratios in membranes composed of L-valine-co-L-phenylalanine poly (ester urea) or L-valine-co-L-phenylalanine poly (ester urea) blended with one or more stabilizing polymers may be used as optimized for the specific application in which the membrane is used.

In some preferred implementations, the surface erosion and degradation mechanisms for the disclosed membranes are sufficiently predictable to allow resorption of the membranes within a specified time range under physiological conditions when used in soft tissue repair applications in humans. In some implementations, the membrane resorbs in an amount of time between 45 and 95 days inclusive under physiological conditions, preferably between 45 and 75 days inclusive under physiological conditions. In some implementations, the membrane resorbs in an amount of time between 95 and 145 days inclusive under physiological conditions, preferably between 105 and 135 days inclusive under physiological conditions. In some implementations, the membrane resorbs in an amount of time between 145 and 195 days inclusive under physiological conditions, preferably between 165 and 195 days inclusive under physiological conditions.

In some implementations, use of the disclosed electrospun membranes in GTR and GBR applications may facilitate osscointegration of dental implants placed with trans-mucosal healing elements immediately into tooth extraction sites. The membrane may preferably comprise an absorbable circumferential membrane arranged to exclude epithelial cells but not osteoblasts from the tooth extraction socket in which a dental implant is placed. As a result of this arrangement, the dental implant osseointegrates into the jaw of the patient without interruption from epithelial cells. Further, the soft texture of the electrospun fibers minimizes tissue irritation and therefore minimizes potential inflammation. These properties of fibrous electrospun scaffolds provide significant advantages over other GTR and GBR scaffolds for use in periodontal GTR and GBR barrier membrane applications.

For wound healing applications, the electrospun membrane/scaffold may be combined with a semi-permeable barrier layer to control water vapor loss, provide a flexible adherent covering for the wound surface, and improve tear strength. The layers may be combined in a single manufacturing step by coating, sequential spinning, or thermal diffusion.

Coating may be achieved, for example, by direct spinning of the membrane/scaffold onto a medical polysiloxane substrate. Altering the spinning distance during the electrospinning process ensures the mechanical integration between the electrospun membrane/scaffold and the silicon layer.

Sequential spinning involves use of electrospinning or electrospraying for sequential layering of the wound membrane/scaffold with a non-resorbable matrix composed of one or more thermoplastic urethanes as a barrier layer. The non-resorbable matrix may be layered onto the electrospun membrane or alternatively the electrospun membrane/scaffold may be layered onto the non-resorbable matrix.

Thermal diffusion involves sequentially electrospinning two layers with a barrier layer of low melting temperature, then applying heat to transform the barrier layer into a film.

In some implementations, the optimal degradation rate for membranes may be between four weeks and six months, depending on the clinical application in which the membranes are used. Increasing surface hydrophilicity and controlling the degradation rate of electrospun biodegradable materials is thus highly desirable for both barrier membrane and scaffold applications. In some implementations, plasma treatment of electrospun biodegradable materials may be used to introduce polar functional groups on the surface of the materials, thereby increasing the hydrophilicity of the surface. In some implementations, the surface of the electrospun biodegradable material is first exposed to a gas at low pressure and then electrically stimulated to ignite the gas, thereby altering the surface chemistry of the material.

In some implementations, the electrospun membranes may preferably exhibit antimicrobial activity.

The disclosed polymer membranes may be treated with an anti-pathogenic agent such as an antiviral agent selected from the group consisting of graphene, nanoparticles, nanocomposites, multivalent metallic ions, and medicinal or other extracts from natural products. The graphene may be functionalized or non-functionalized. The nanoparticles may preferably be metal nanoparticles such as silver nanoparticles or zinc nanoparticles. The nanocomposites may preferably be silver-doped titanium dioxide nanomaterials. The multivalent metallic ions may preferably be metal ions such as Cu²⁺ or Zn²⁺ cations. The extracts from natural products may preferably be licorice extracts.

Alternatively, an anti-pathogenic agent such as an antiviral agent selected from the group consisting of graphene, nanoparticles, nanocomposites, multivalent metallic ions, and medicinal or other extracts from natural products may be incorporated into the electrospun membrane by adding it into the polymer solution that is subsequently electrospun. The graphene may be functionalized or non-functionalized. The nanoparticles may preferably be metal nanoparticles such as silver nanoparticles or zinc nanoparticles. The nanocomposites may preferably be silver-doped titanium dioxide nanomaterials. The multivalent metallic ions may preferably be metal ions such as Cu²⁺ or Zn²⁺ cations. The extracts from natural products may preferably be licorice extracts.

In some implementations, growth factors may be incorporated into the disclosed electrospun biodegradable materials. The incorporation of growth factors into electrospun matrices for tissue engineering may enhance bioactivity by supplying appropriate physical and chemical cues to promote cellular proliferation and migration, thereby increasing the cellularization of the structures. The electrospun membrane may replicate the role of the native ECM in normal wound healing by serving as a reservoir of soluble growth factors critical to regeneration and providing a template for tissue repair. This may accelerate cellularization and tissue repair.

In some implementations, platelet-rich plasma (PRP) therapy may be incorporated with electrospun polymeric membranes to harness the reparative potential and bioactivity found in a platelet-rich plasma. PRP therapy is a method of collecting and concentrating autologous platelets, through centrifugation and isolation, for the purpose of activating and releasing their dense, growth factor-rich granules. The discharge of these concentrated granules releases a number of growth factors and cytokines in physiologically relevant ratios, albeit in concentrations several times higher than that of normal blood, that are critical to tissue regeneration and cellular recruitment. Clinically, PRP therapy has been used to stimulate tissue growth and regeneration in a number of different tissues, effectively accelerating the healing response in patients suffering from osteochondral defects. The combination of a PRP with an electrospun polymeric membrane scaffold may generate a product that will provide the advantages of using electrospun biodegradable materials as a barrier for the epithelial layer in periodontium and the enhanced healing and regeneration of bone tissues by the sustained release of the PRP component.

A method of regenerating bone or tissue in a patient, wherein the method comprises applying a membrane selected from the group consisting of the membranes described herein into a cavity or opening requiring soft tissue repair, is also disclosed herein.

The following example is provided as a specific illustration of the disclosed methods and products. It should be understood, however, that the invention is not limited to the specific details set forth in the example.

Further, any range of numbers recited above or in the paragraphs hereinafter describing or claiming various aspects of the invention, such as ranges that represent a particular set of properties, units of measure, conditions, physical states, or percentages, is intended to literally incorporate any number falling within such range, including any subset of numbers or ranges subsumed within any range so recited. The term “about” when used as a modifier is intended to convey that the numbers and ranges disclosed herein may be flexible as understood by ordinarily skilled artisans and that practice of the disclosed invention by ordinarily skilled artisans using properties that are outside of a literal range will achieve the desired result.

### Materials and Methods

Materials: Amino acid-based poly (ester urea) poly (l-PHE-6) with a molecular weight of 45 kDa (hereinafter “PEU”) was provided by Prof. Matthew Becker at the University of Akron. Hexafluoroisopropanol (HFIP) was purchased from Oakwood Products Inc., Estill, SC.

Solution Preparation: PEU was added to HFIP to generate 7% and 15% wt/vol solutions. The solutions were mixed on a stirring plate until the polymer pellets completely dissolved.

Membrane Fabrication: A bilayer membrane was produced via an electrospinning process as described. The prepared PEU solution was added to a syringe and loaded in a syringe pump connected to an electrospinning machine. The electrospinning setup included a programmable syringe pump (Model R99-E, Razel Scientific Instruments) attached to a glass syringe with a flat-end needle connected to a positive terminal of a high voltage power supply (0-30 kV) (EN 61010-1, Glassman High Voltage). The fibers were collected on a rotating aluminum mandrel with a 25 mm diameter at a rotation speed of 900 rpm.

A bilayer membrane was produced using the following procedure. First, the 15% solution was electrospun at a flow rate of 8 mL/h and an applied voltage of 13 kV. An 18 gauge needle was used, and the distance between the tip of the needle and the rotating mandrel was set to 20 cm. Second, the 7% solution was electrospun at a flow rate of 3 mL/h and an applied voltage of 16 kV. A 21 gauge needle was used, and the distance between the tip of the needle and the rotating mandrel was set to 25 cm. A total of 4 mL of the polymer solution was dispensed for each layer. The time between spinning processes between the two layers was less than ten minutes. The two layers were also electrospun separately for the purpose of characterizing the individual layers.

Post-Fabrication Treatment: The electrospun tube generated was dried on the mandrel in a vacuum oven at 35 degrees Celsius for 20 h to remove residual solvent. After drying, the membrane was released off the mandrel and the electrospun tube was cut into a flat sheet. The sheet was then exposed to oxygen plasma surface treatment using a low-pressure plasma system (Diener FEMTO plasma system, Model FEMTO40KHZ) at a flow rate of 70% and applied energy of 30%. The electrospun sheet was then cut into multiple membranes of 20×30 mm using scissors. The membranes were then sterilized via E-beam at 30 kGy (Steri-Tek, Fremont, CA).

### Analyses

Morphology Analysis: The morphology of the electrospun membranes were analyzed by scanning electron microscopy (Zeiss, SUPRA 55VP). Membrane samples were sputter coated with platinum and palladium using a sputter coater for two minutes (Quorum Technologies, EMS 300T Dual Head) under a pressure of 8×10⁻² mbar and an electric potential of 300 V.

Fiber Diameter Analysis: Fiber diameters were measured from SEM images using analysis software (FibraQuant 1.3.153, NanoScaffold Technologies, Chapel Hill, NC). At least 250 measurements were recorded on each scaffold type using top view SEM images of 2000×. These measurements were reviewed by an operator to confirm program accuracy.

Porosity Analysis: The porosity of the membranes was evaluated using a gravimetric measurement method. Using this method, porosity (a) is defined in terms of the apparent density of the fiber mat (PAPP) and bulk density of the polymer (Pmaterial) of which it is made:

$$ε = 1 - {ρ}_{APP} / {ρ}_{material}$$

The apparent scaffold density β_{APP} was measured as a mass to volume ratio on 10 mm dry disks:

$${ρ}_{APP} = mass / {V}_{material}$$

Pore Size Analysis: The pore size of the membranes was estimated indirectly through approximate statistical models. See Kim, C. H., et al. J. Biomed. Mater. Res. Part B Appl. Biomater. 2006, 78B, 283; Eichhorn, S. J., et al. “Statistical Geometry of Pores and Statistics of Porous Nanofibrous Assemblies,” J. R. Soc. Interface, 2005, 2, 309-18. The model yields the following approximated distribution p(r) of 3D pore radii r associated with a unimodal fiber distribution:

$$p ( r ) = \frac{n}{ε} {( \frac{Γ ( k , bn )}{Γ ( k )} )}^{n / ε1} \frac{{b}^{k}}{Γ ( k )} {r}^{k - 1} exp ( - br )$$

where Γ(k,bn) and Γ(k) are the incomplete and complete gamma functions respectively, k is a constant parameter equal to 1.6, n is an equivalent number of layers, and b is an experimental parameter.

The experimental parameter is defined as b=2k/ [symbol] r_{2D} [symbol] , a function of the average bidimensional pore diameter [symbol] r_{2D} [symbol] of one fiber layer, which in turn is related to ε and to the average [symbol] ω [symbol] by

$$〈 {r}_{2 D} 〉 ≅ \frac{\sqrt{π}}{4} ( \frac{π}{2 ln ( 1 / ε )} - 1 ) 〈 ω 〉$$

The distribution p(r) is conceived as the superposition of 2D layers, the number n of which was assumed to be:

$$n ( ε , C ) ≅ \frac{c}{ln ( 1 / ε )}$$

where the coverage parameter c is defined as:

$$c ( β , 〈 ω 〉 ) = \frac{total apparent scaffold volume}{volume of 1 - monolayer of fibers} ≅ \frac{4 β}{π {ρ}_{PEU} 〈 ω 〉}$$

The coverage parameter corresponds to the average surface density, namely the ratio of the mass of the 20 mm disks and their surface area. Hence, the distribution p(r) is determined by inputting the set { [symbol] ω [symbol] , ε, β} of three experimentally-determined input parameters, and the average pore radius [symbol] r [symbol] , taken as the representative measure for the scaffold, is simply

$$〈 r 〉 = {\int }_{0}^{∞} rp ( r ) dr$$

A second model, see Sampson, W. W. “Modeling Stochastic Fibrous Materials with Mathematica,” In Engineering Materials and Processes, XII, Berlin: Springer, 2009, was also employed to obtain a refined estimate for [symbol] r [symbol] . The second model differs from the former only with respect to the definition of [symbol] r_{2D} [symbol] .

$$〈 {r}_{2 D} 〉 ≅ \frac{〈 ω 〉}{ln ( 1 / ε )}$$

Both models were implemented in MAPLE (Maplesoft, Ontario, Canada) for symbolic computation.

Wettability Analysis: Wetting experiments were performed using a Cahn model 315 Dynamic Contact Angle (DCA) analyzer with WinDCA 32 v. 2.11 software. A sample was lowered into a test fluid, partially immersed in the fluid, and then withdrawn from the test fluid. The instrument records the changes in mass sensed by the balance as the process plots the changes as a function of sample position. The DCA wicking experiments were conducted using 10 mm wide specimens of the membrane samples. The instrument was programmed at a speed of 80 μm/s.

Mechanical Analysis: Tensile testing was performed on 10 mm×50 mm samples that were mounted on an electromechanical load frame (Shimadzu AGS-X electromechanical load frame) using a 1 kN load cell. The testing parameters were the same for all samples, using a data acquisition rate of 100 Hz, a gauge length of 30 mm, and a test speed of 1 mm/s.

### Results

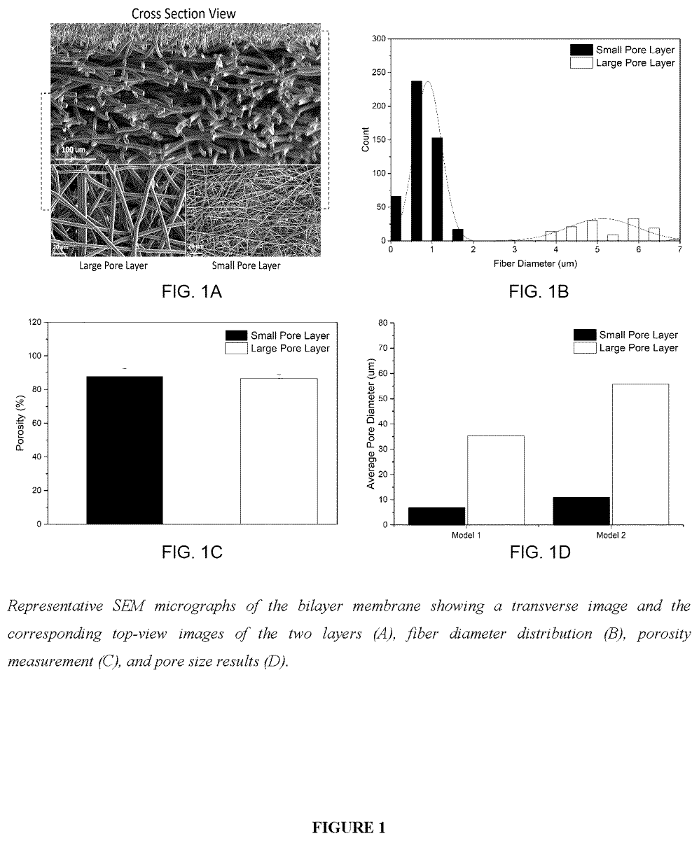

Morphology and Microstructure Characteristics: Representative SEM micrographs of cross-section and top-views of a representative membrane sample are shown in FIG. 1A. In the cross-section, the fibers comprising each layer were discernable. The structure of the exterior side of the membrane (small pore layer) was composed of a smaller fiber diameter than the interior side of the membrane (large pore layer). The top-view images of both exterior and interior sides show that fibers were smooth and randomly oriented while no beads were observed. The thickness of the individual layers was about 50 μm and 300 μm for the small pore layer and large pore layer respectively. Based on the FibraQuant analysis, the average fiber diameter for the individual layers was 0.89 μm and 5.12 μm for the small pore layer and large pore layer respectively. The fiber diameter distribution is shown in FIG. 1B. Based on the gravimetric measurements, the average porosity was 87.9% and 86.6% for the small pore layer and large pore layer respectively, as shown in FIG. 1C. The average pore diameter for the individual layers was calculated by mathematical models. The models demonstrated that the layer constructed using fibers having a small fiber diameter had narrower pores than the layer constructed using fibers having a large fiber diameter. The average pore size for the small pore layer was 6.9 μm and 10.9 μm based on the first and second mathematical models described above, respectively. The average pore size for the large pore layer was 35.3 μm and 55.8 μm based on the first and second mathematical models described above, respectively.

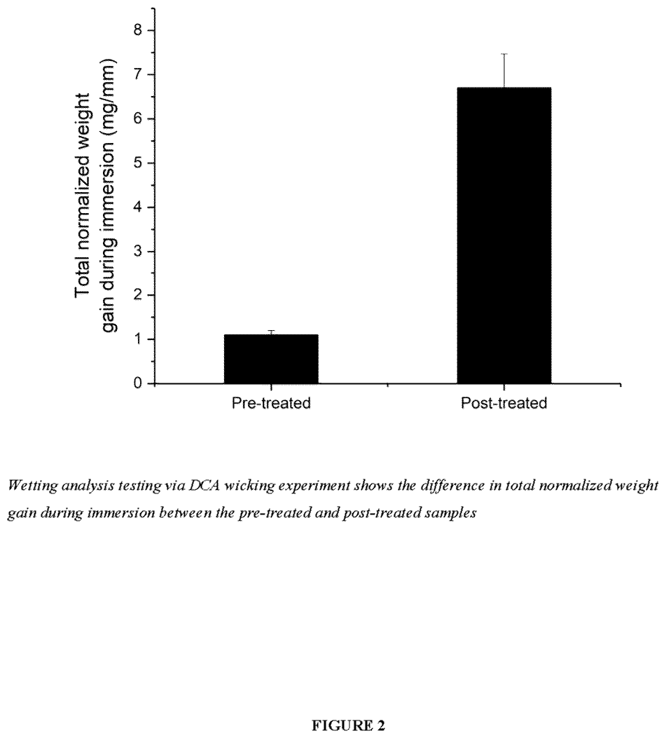

Wettability Characteristics: The effect of plasma surface treatment on the membrane's hydrophilicity was evaluated via a DCA wicking experiment. Both pre-treated and post-treated samples were tested. The total normalized weight gain during samples immersion was 1.1 mg/mm for the pre-treated sample and 6.8 mg/mm for the post-treated sample, as shown in FIG. 2.

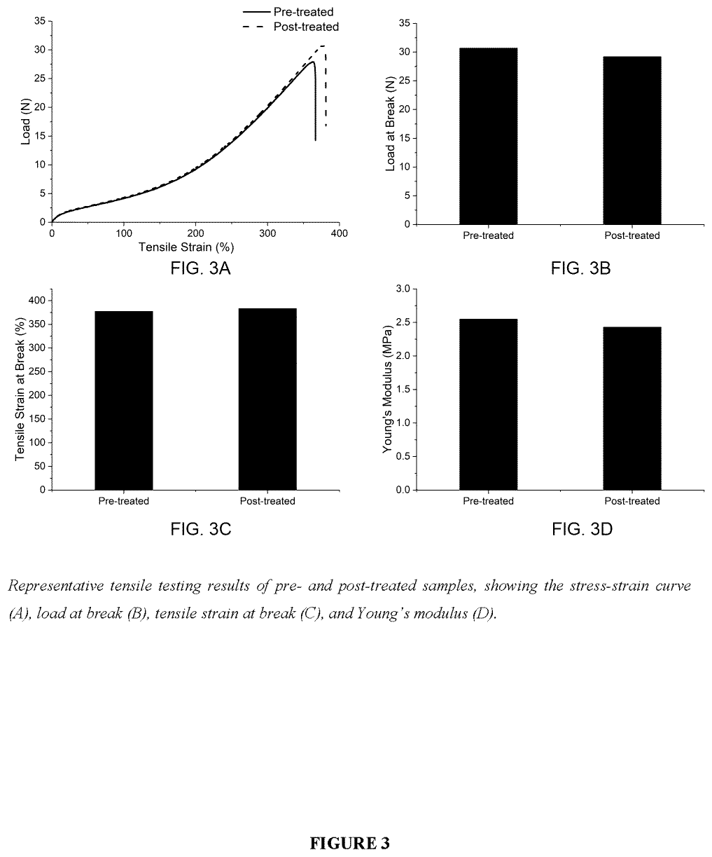

Mechanical Characteristics: The tensile strength of the membrane samples was evaluated. The tensile properties of both pre-plasma and post-plasma samples were almost identical, as shown in FIG. 3A. Membrane samples had an ultimate tensile strength of 30 N as shown in FIG. 3B, a tensile strain at break above 350% as shown in FIG. 3C, and a Young's modulus of about 2.5 MPa as shown in FIG. 3D.

### DISCUSSION

In electrospun constructs, average pore size is not an independent design parameter, but it is dependent on other microstructure characteristics. Among these microstructure characteristics, fiber diameter has the most significant impact on average pore size. Thus, to generate a multi-layer membrane with different average pore sizes for each of the layers, it is necessary to generate fibers with different diameters. Adjusting the viscosity of the solutions used for electrospinning allowed the generation of fibers with different diameters. In addition, adjustments to process parameters such as applied voltage, needle gauge size, and screen distance generated better results.

The electrospun fibers generated were smooth, without bead formation or other morphological defects. Moreover, the differences between small average pore size and large average pore size layers of the membranes generated were readily distinguishable by SEM. The fiber diameter of the large pore layer was significantly larger than the small pore layer, and the fiber diameter measurements exhibited narrow size distribution for both layers. This indicates that the spinning jet used in electrospinning was stable.

The non-woven structure produced by electrospinning usually features high surface area to volume ratio regardless of fiber diameter. Consistent with this expected result, the average porosity shown in FIG. 1C did not show significant differences between the large average pore size and small average pore size layers.

Wettability testing demonstrated that the hydrophilicity of the membranes improved dramatically after plasma treatment. Membrane hydrophilicity is critical for use as a dental barrier membrane, as this enhances conformability during placement of the membrane in a surgical site and facilitates handling of the membrane during surgery.

Mechanical testing showing that the tensile properties of the bilayer membrane were far superior to those of currently available barrier membranes. Most importantly, undesirable delamination was not observed even at high strain conditions. This indicates that the two layers of the membrane were fully integrated and cannot be separated even under high tensile stress conditions.

### Antimicrobial Activity

Electrospun fibers generated according to the methods disclosed above were evaluated for antimicrobial activity. Electrospun fiber samples were evaluated in a study based on ASTM E2315 “Standard Guide for Assessment of Antimicrobial Activity Using a Time-Kill Procedure.”

Four fibers were exposed to 100 μL of dilutions between about 10-3 and 105 of E. coli for three hours, incubated overnight at 37° C., and counted the next day. No bacterial colonies were observed.

### Numbered Examples

The following numbered examples provide exemplary illustrations of the disclosed methods and products. It should be understood, however, that the invention is not limited to the specific details set forth in the examples.

Example 1: A membrane suitable for use soft tissue repair applications composed of two or more polymer layers is disclosed. The two or more polymer layers each comprise a polymer or a combination of polymers, where the two or more polymer layers comprise a first polymer layer and a second polymer layer. The first polymer layer comprises a first set of polymer fibers, where at least 95% of the first set of polymer fibers have fiber diameters of about 0.1-2 μm, and where the first polymer layer has an average pore diameter of about 5-15 μm. The second polymer layer comprises a second set of polymer fibers, where at least 95% of the second set of polymer fibers have fiber diameters of about 3-7 μm, and where the second polymer layer has an average pore diameter of about 35-60 μm. The porosities of the first polymer layer and the second polymer layer differ by less than 5%, and the porosities of both the first polymer layer and the second polymer layer are about 80-95%. The two or more polymer layers are generated by one or more steps of electrospinning a solution containing a polymer or combination of polymers in a solvent to generate electrospun polymer fibers.

Example 2: The membrane of Example 1 where the polymer or combination of polymers includes an L-valine-co-L-phenylalanine poly (ester urea) is disclosed. The solvent is hexafluoroisopropanol, and the two or more polymer layers are the same polymer or combination of polymers.

Example 3: The membrane of Example 1 where mechanical integration and binding between the polymer layers is enhanced by electrospinning wet fibers before electrospinning the subsequent polymer layer is disclosed.

Example 4: The membrane of Example 1, where the one or more steps of electrospinning the polymer or combination of polymers are implemented using an electrospinning apparatus comprising a syringe pump, a syringe, a power supply, and a mandrel or drum, is disclosed. A tubular braided structure or collapsible sleeve is applied to the mandrel or drum before electrospinning. The membrane has a first surface and a second surface, where the first surface is proximate to the braid or sleeve during electrospinning, and the electrospun polymer fibers at the first surface are substantially undamaged after release of the membrane from the mandrel.

Example 5: The membrane of Example 1, where the membrane is surface-modified by blending the solution with a non-ionic surfactant, is disclosed.

Example 6: The membrane of Example 1, where an additive or coating material is added to the solution or physically coated on at least one surface of the membrane after all of the electrospinning steps are completed, is disclosed.

Example 7: The membrane of Example 1, where at least one surface of the membrane is coated with an adhesive, is disclosed.

Example 8: The membrane of Example 7, where the adhesive is one or more adhesives selected from the group consisting of poly (dopamine), fibrin glue, elastin, dihydroxyphenylalanine (DOPA) derivatives, polyethylene glycol (PEG), hyaluronic acid, polyethylene glycol (PEG) and its derivatives, alginate, calcium, gelatin, chitosan, polysaccharides, and poly amido amine (PAMAM) dendrimer, is disclosed.

Example 9: The membrane of Example 1, where the membrane resorbs in an amount of time between 45 and 75 days under physiological conditions, is disclosed.

Example 10: The membrane of Example 1, where the membrane resorbs in an amount of time between 105 and 135 days under physiological conditions, is disclosed.

Example 11: The membrane of Example 1, where the membrane resorbs in an amount of time between 165 and 195 days under physiological conditions, is disclosed.

Example 12: The membrane of Example 1, where the membrane resorbs in an amount of time between 45 and 195 days under physiological conditions, is disclosed.

Example 13: The membrane of Example 1, where the membrane exhibits antimicrobial activity, is disclosed.

Example 14: The membrane of Example 1, where the polymer or combination of polymers further comprises polydioxanone, is disclosed.

Example 15: The membrane of Example 2, where the polymer or combination of polymers further comprises polydioxanone, is disclosed.

Example 16: The membrane of Example 4, where the polymer or combination of polymers further comprises polydioxanone, is disclosed.

Example 17: The membrane of Example 1, where the membrane is further composed of a third polymer layer, is disclosed. The first polymer layer is positioned between the second polymer layer and the third polymer layer. The third polymer layer has an average pore diameter and porosity that is the same as the second polymer layer, and the third polymer layer is composed of a third set of polymer fibers that has the same distribution of fiber diameters as the second set of polymer fibers of the second polymer layer.

Example 18: The membrane of Example 2, where the membrane is further composed of a third polymer layer, is disclosed. The first polymer layer is positioned between the second polymer layer and the third polymer layer. The third polymer layer has an average pore diameter and porosity that is the same as the second polymer layer, and the third polymer layer is composed of a third set of polymer fibers that has the same distribution of fiber diameters as the second set of polymer fibers of the second polymer layer.

Example 19: The membrane of Example 4, where the membrane is further composed of a third polymer layer, is disclosed. The first polymer layer is positioned between the second polymer layer and the third polymer layer. The third polymer layer has an average pore diameter and porosity that is the same as the second polymer layer, and the third polymer layer is composed of a third set of polymer fibers that has the same distribution of fiber diameters as the second set of polymer fibers of the second polymer layer.

Example 20: The membrane of Example 15, where the membrane is further composed of a third polymer layer, is disclosed. The first polymer layer is positioned between the second polymer layer and the third polymer layer. The third polymer layer has an average pore diameter and porosity that is the same as the second polymer layer, and the third polymer layer is composed of a third set of polymer fibers that has the same distribution of fiber diameters as the second set of polymer fibers of the second polymer layer.

The previous description of the disclosed embodiments is provided to enable any person skilled in the art to make or use the invention disclosed herein. Although the various inventive aspects are disclosed in the context of certain illustrated embodiments, implementations, and examples, it should be understood by those skilled in the art that the invention extends beyond the specifically disclosed embodiments to other alternative embodiments and/or uses of the invention and obvious modifications and equivalents thereof. In addition, while a number of variations of various inventive aspects have been shown and described in detail, other modifications that are within their scope will be readily apparent to those skilled in the art based upon reviewing this disclosure. It should be also understood that the scope of this disclosure includes the various combinations or sub-combinations of the specific features and aspects of the embodiments disclosed herein, such that the various features, modes of implementation, and aspects of the disclosed subject matter may be combined with or substituted for one another. The generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

Each of the foregoing and various aspects, together with those summarized above or otherwise disclosed herein, including the figures, may be combined without limitation to form claims for a device, apparatus, system, method of manufacture, and/or method of use.

All references cited herein are hereby expressly incorporated by reference.

What is claimed is:

1. A membrane suitable for use soft tissue repair applications comprising two or more polymer layers;
wherein the two or more polymer layers each comprise a polymer or a combination of polymers;
wherein the two or more polymer layers comprise a first polymer layer and a second polymer layer;
wherein the first polymer layer comprises a first set of polymer fibers,
wherein at least 95% of the first set of polymer fibers have fiber diameters of about 0.1-2 μm;
wherein the first polymer layer has an average pore diameter of about 5-15 μm;
wherein the second polymer layer comprises a second set of polymer fibers,
wherein at least 95% of the second set of polymer fibers have fiber diameters of about 3-7 μm;
wherein the second polymer layer has an average pore diameter of about 35-60 μm;
wherein the porosities of the first polymer layer and the second polymer layer differ by less than 5%;
wherein the porosity of the first polymer layer is about 80-95% and the porosity of the second polymer layer is about 80-95%; and
wherein the two or more polymer layers are generated by one or more steps of electrospinning a solution containing a polymer or combination of polymers in a solvent to generate electrospun polymer fibers.

2. The membrane of claim 1,
wherein the polymer or combination of polymers comprises an L-valine-co-L-phenylalanine poly (ester urea);
wherein the solvent is hexafluoroisopropanol; and
wherein the two or more polymer layers comprise the same polymer or combination of polymers.

3. The membrane of claim 1, wherein mechanical integration and binding between the polymer layers is enhanced by electrospinning wet fibers before electrospinning the subsequent polymer layer.

4. The membrane of claim 1,
wherein the one or more steps of electrospinning the polymer or combination of polymers are implemented using an electrospinning apparatus comprising a syringe pump, a syringe, a power supply, and a mandrel or drum;
wherein a tubular braided structure or collapsible sleeve is applied to the mandrel or drum before electrospinning;
wherein the membrane has a first surface and a second surface;
wherein the first surface is proximate to the braid or sleeve during electrospinning; and
wherein the electrospun polymer fibers at the first surface are substantially undamaged after release of the membrane from the mandrel.

5. The membrane of claim 1, wherein the membrane is surface-modified by blending the solution with a non-ionic surfactant.

6. The membrane of claim 1, wherein an additive or coating material is added to the solution or physically coated on at least one surface of the membrane after all of the electrospinning steps are completed.

7. The membrane of claim 1, wherein at least one surface of the membrane is coated with an adhesive.

8. The membrane of claim 7, wherein the adhesive is one or more adhesives selected from the group consisting of poly (dopamine), dihydroxyphenylalanine (DOPA) derivatives, polyethylene glycol (PEG), hyaluronic acid, polyethylene glycol (PEG) and its derivatives, alginate, calcium, gelatin, chitosan, polysaccharides, and poly amido amine (PAMAM) dendrimer.

9. The membrane of claim 1, wherein the membrane resorbs in an amount of time between 45 and 75 days under physiological conditions.

10. The membrane of claim 1, wherein the membrane resorbs in an amount of time between 105 and 135 days under physiological conditions.

11. The membrane of claim 1, wherein the membrane resorbs in an amount of time between 165 and 195 days under physiological conditions.

12. The membrane of claim 1, wherein the membrane resorbs in an amount of time between 45 and 195 days under physiological conditions.

13. The membrane of claim 1, wherein the membrane exhibits antimicrobial activity.

14. The membrane of claim 1, wherein the polymer or combination of polymers further comprises polydioxanone.

15. The membrane of claim 2, wherein the polymer or combination of polymers further comprises polydioxanone.

16. The membrane of claim 4, wherein the polymer or combination of polymers further comprises polydioxanone.

17. The membrane of claim 1, wherein the membrane further comprises a third polymer layer,
wherein the first polymer layer is positioned between the second polymer layer and the third polymer layer,
wherein the third polymer layer has an average pore diameter and porosity that is the same as the second polymer layer, and
wherein the third polymer layer comprises a third set of polymer fibers that has the same distribution of fiber diameters as the second set of polymer fibers of the second polymer layer.

18. The membrane of claim 2, wherein the membrane further comprises a third polymer layer,
wherein the first polymer layer is positioned between the second polymer layer and the third polymer layer,
wherein the third polymer layer has an average pore diameter and porosity that is the same as the second polymer layer, and
wherein the third polymer layer comprises a third set of polymer fibers that has the same distribution of fiber diameters as the second set of polymer fibers of the second polymer layer.

19. The membrane of claim 4, wherein the membrane further comprises a third polymer layer,
wherein the first polymer layer is positioned between the second polymer layer and the third polymer layer,
wherein the third polymer layer has an average pore diameter and porosity that is the same as the second polymer layer, and
wherein the third polymer layer comprises a third set of polymer fibers that has the same distribution of fiber diameters as the second set of polymer fibers of the second polymer layer.

20. The membrane of claim 15, wherein the membrane further comprises a third polymer layer,
wherein the first polymer layer is positioned between the second polymer layer and the third polymer layer,
wherein the third polymer layer has an average pore diameter and porosity that is the same as the second polymer layer, and
wherein the third polymer layer comprises a third set of polymer fibers that has the same distribution of fiber diameters as the second set of polymer fibers of the second polymer layer.
